(19) United States
(12) Patent Application Publication
(10) Pub. No.: US 2021/0393385 A1
(43) Pub. Date: Dec. 23, 2021

(54) DENTAL APPLIANCE WITH ION EXCHANGE COATING

(71) Applicant: 3M INNOVATIVE PROPERTIES COMPANY, St. Paul55133-3427, MN (US)

(72) Inventors: Zeba Parkar, Marietta, GA (US); Victor Ho, Saint Paul, MN (US); Joel D. Oxman, Minneapolis, MN (US); Jason A. Bender, Woodbury, MN (US)

(21) Appl. No.: 17/292,847

(22) PCT Filed: Nov. 19, 2019
(86) PCT No.: PCT/IB2019/059904

§ 371 Date: May 11, 2021

Related U.S. Application Data
(60) Provisional application No. 62/769,540, filed on Nov. 19, 2018.

(51) Int. Cl. A61C 19/06; A61C 7/08; A61C 19/02; A61K 6/20; A61K 6/70

(52) U.S. Cl. CPC A61C 19/063; A61C 7/08; A61K 6/70; A61K 6/20; A61C 19/02

(57) ABSTRACT

A dental appliance including a polymeric shell with an arrangement of cavities configured to receive one or more teeth and a coating on at least portion of the polymeric shell. The coating can include a polyester with an ion exchange functional group covalently bonded thereto, and the ion exchange functional group can release in an oral environment at least one therapeutically beneficial ion at a surface of a tooth.

### BACKGROUND

Orthodontic treatments involve repositioning misaligned teeth and improving bite configurations for improved cosmetic appearance and dental function. Repositioning teeth is accomplished by applying controlled forces to the teeth over an extended time period.

Teeth may be repositioned by placing a polymeric incremental position adjustment appliance, generally referred to as an orthodontic aligner or an orthodontic aligner tray, over the teeth of the patient for each treatment stage of an orthodontic treatment. The orthodontic alignment trays include a polymeric shell with a plurality of cavities configured to receive one or more teeth. The individual cavities in the polymeric shell are shaped to exert force on one or more teeth to resiliently and incrementally reposition selected teeth or groups of teeth in the upper or lower jaw. A series of orthodontic aligner trays are provided for wear by a patient sequentially and alternatingly during each stage of the orthodontic treatment to gradually reposition teeth from one tooth arrangement to a successive tooth arrangement to achieve a desired tooth alignment condition. Once the desired alignment condition is achieved, an aligner tray, or a series of aligner trays, may be used periodically or continuously in the mouth of the patient to maintain tooth alignment. In addition, orthodontic retainer trays may be used for an extended time period to maintain tooth alignment following the initial orthodontic treatment. Mouthguards and nightguards may also be used to temporarily protect teeth during athletic activities or to prevent damage caused by tooth-to-tooth contact or rubbing.

A stage of orthodontic treatment may require that a dental appliance remain in the mouth of the patient for up to 22 hours a day, over an extended time period of days, weeks or even months.

Saliva is the mouth's primary defense against tooth decay. Healthy saliva flow helps prevent cavities by physically removing bacteria from the oral cavity before they can become attached to tooth and tissue surfaces and form a protected biofilm. The flow of saliva also helps dilute sugars and acids introduced by intake of food and beverages. The buffering capacity of saliva neutralizes acids and aids in the digestive process.

While the orthodontic retainer or aligner tray is in use in the mouth of the patient, acidic species derived from one or more of bacteria, sugars, foods, and beverages may be confined between the aligner tray and the teeth. The presence of these compounds against the surfaces of the teeth can demineralize dental hard tissues that include, for example, calcium phosphate entities such as crystalline calcium hydroxyapatite. The increasing use of orthodontic aligners can potentially trap the acidogenic agents and/or acids on the surfaces of the teeth for extended periods of time, which can increase demineralization of the tooth enamel.

Demineralization can be minimized by neutralizing acids at the surface of the teeth with, for example, bases or buffering agents. Demineralization can also be reduced by introducing ions such as calcium and/or phosphate to maintain a positive equilibrium or to facilitate remineralization. Fluoride can also react with calcium based minerals to form calcium fluoride or calcium fluoroapatite that is far less soluble in the presence of low pH.

### SUMMARY

Placement of a dental appliance such as, for example, an orthodontic aligner tray, a retainer tray, a mouthguard, a nightguard, and the like, over the teeth of a patient can impede the natural flow of saliva around the teeth, which in some cases may increase the risk of tooth decay, particularly if the patient fails to consistently follow recommended regimens for tray cleaning and tooth brushing.

In one aspect, the present disclosure is directed to a dental appliance that includes an ion exchange coating layer configured to supply therapeutically beneficial ions to the surface of the teeth of a patient. For example, the ion exchange coating layer can supply ions to more effectively control pH at the surface of the teeth to neutralize acids, or can introduce calcium and/or phosphate into the spaces between the orthodontic aligner tray and the teeth to maintain a positive equilibrium at the surface of the teeth and facilitate remineralization and/or minimize demineralization. The ion exchange coating layer on the dental appliance can also supply fluoride at the surface of the teeth, which can react with calcium based minerals to form calcium fluoride or calcium fluoroapatite that is far less soluble in the presence of low pH. The ion exchange coated dental appliance can reduce the risk or mitigate demineralization during the extended use of the dental appliance in the mouth of a patient.

In one example, a dental appliance includes a polymeric shell with an arrangement of cavities configured to receive one or more teeth and a coating on at least portion of the polymeric shell. The coating can include a polyester with an ion exchange functional group covalently bonded thereto, and the ion exchange functional group can release in an oral environment at least one therapeutically beneficial ion at a surface of a tooth.

Another example includes a method of making a dental appliance. The method comprises forming a polymeric shell comprising a plurality of cavities in a first major surface thereof, wherein the cavities are configured to receive one or more teeth. The method further comprises applying a coating composition on the polymeric shell, wherein the coating composition comprises a polyester with an ion exchange functional group covalently bonded thereto, wherein the ion exchange functional group is configurable to release in an oral environment at least one therapeutically beneficial ion at a surface of a tooth, and wherein the ion exchange functional group comprises a first metal ion.

Another example includes a method of making a dental appliance. The method comprises applying a coating composition on at least one major surface of a substantially flat sheet of a polymeric material, wherein the coating composition comprises a polyester with an ion exchange functional group covalently bonded thereto, wherein the ion exchange functional group is configurable to release in an oral environment at least one therapeutically beneficial ion at a surface of a tooth, and wherein the ion exchange functional group comprises a first metal ion. The method further comprises forming a plurality of cavities in the polymeric material to form a polymeric shell, wherein the cavities are configured to receive one or more teeth.

Another example includes a method of making a dental appliance. The method comprises applying a coating composition on at least one major surface of a substantially flat sheet of a polymeric material, wherein the coating composition comprises a polymer with an ion exchange functional group covalently bonded thereto, wherein the polymer comprises a polyester, wherein the polymer comprises a quaternary ammonium compound chosen from tetraalkylammoniums, alkylated pyridines, alkylated immidazoles, and combinations thereof, and wherein the ion exchange functional group is configurable to release in an oral environment at least one therapeutically beneficial ion at a surface of a tooth, and wherein the ion exchange functional group comprises a first metal ion. The method further comprises forming a plurality of cavities in the polymeric material to form a polymeric shell, wherein the cavities are configured to receive one or more teeth.

Another example includes a method of treating demineralization of a surface of a tooth. The method comprises positioning a dental appliance adjacent to the surface of the tooth, wherein the dental appliance comprises a polymeric shell with a plurality of cavities configured to incrementally move one or more teeth, and wherein the polymeric shell comprises a coating thereon. The coating comprises a sulfopolyester with an ion exchange functional group covalently bonded thereto, and wherein the ion exchange functional group supplies in an oral environment at least one therapeutically beneficial ion at a surface of a tooth.

Another example includes a method of treating demineralization of a tooth. The method comprises providing a polymeric shell with a plurality of cavities configured to incrementally move one or more teeth, wherein the polymeric shell comprises a coating thereon, the coating comprising a sulfopolyester with an ion exchange functional group, wherein the ion exchange functional group comprises a first metal ion; applying to the first coating an ionic solution with a second metal ion, different from the first metal ion; replacing the first metal ion on at least a portion of the ion exchange functional group with the second metal ion to form a second coating, wherein the second ion is a therapeutically beneficial ion chosen from calcium, fluoride, phosphate, and combinations thereof; and positioning the second coating such that the second coating is adjacent to a surface of at least one tooth, the second coating releasing in an oral environment at least one therapeutically beneficial ion at the surface of the tooth.

Another example includes a kit. The kit comprises a dental appliance and a solution. The dental appliance comprises a polymeric shell with a plurality of cavities configured to incrementally move one or more teeth, wherein the polymeric shell comprises a coating thereon, the coating comprising a sulfopolyester with an ion exchange functional group covalently bonded thereto, and wherein the ion exchange functional group releases in an oral environment at least one therapeutically beneficial ion at a surface of a tooth. The solution comprises ions to periodically replenish the therapeutically beneficial ions of the coating.

Another example includes a dental appliance comprising a polymeric shell with an arrangement of cavities configured to receive one or more teeth; and a coating on at least portion of the polymeric shell. The coating comprises a polymer with an ion exchange functional group covalently bonded thereto, wherein the polymer comprises a quaternary ammonium compound chosen from tetraalkylammoniums, alkylated pyridines, alkylated immidazoles, and combinations thereof. The ion exchange functional group releases in an oral environment at least one therapeutically beneficial ion at a surface of a tooth.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic overhead perspective view of a dental alignment tray;

FIG. 2 is a schematic overhead perspective view of a method for using a dental alignment tray by placing the dental alignment tray to overlie teeth;

FIG. 3 is a schematic diagram of components of a kit including the dental appliance and an ionic aqueous solution;

FIG. 4 is a schematic illustration of a reclosable storage unit and dispenser configured to hold a dental appliance and dispense an ionic aqueous solution into the tooth-retaining cavities in the dental appliance; and

FIG. 5 is a graph illustrating the calculated calcium concentration in ppm for various samples, as detailed in the Examples below.

Like symbols in the drawings indicate like elements.

### DETAILED DESCRIPTION

Referring to FIG. 1, a dental appliance 100 includes a thin polymeric shell 102 with tooth-retaining cavities 104 configured to fit over one or more of the teeth in the upper or lower jaw of a patient. In the embodiment shown in FIG. 1, the dental appliance 100 is an orthodontic aligner tray, but in other embodiments the dental appliance can be, for example, an orthodontic retainer tray, a mouthguard, or a nightguard. In the embodiment of FIG. 1, the tooth-retaining cavities 104 are shaped to receive and resiliently reposition one or more teeth from one tooth arrangement to a successive tooth arrangement. In other embodiments, a dental retainer tray may include tooth-retaining cavities 104 shaped to receive and maintain the position of the previously realigned one or more teeth, while a mouthguard or a nightguard includes tooth-retaining cavities 104 shaped to protect teeth during sports activities or to prevent teeth in the upper and lower jaws from rubbing against one another and causing premature wear to a tooth surface.

Ion exchange is the reversible exchanging of ions between a solid, e.g., an ion exchange layer, and a liquid. The solid in the ion exchange can exchange ions without experiencing a permanent change in the structure of the solid. The first major external surface 106 of the shell 102, or a second major internal surface 108 of the shell 102 that contacts the teeth of the patient, or both, include an ion exchange coating layer 110 that supplies therapeutically beneficial ions in the mouth of the patient to improve oral health. For example, the ion exchange coating layer 110 can supply ions to balance pH in regions adjacent to the surface of the teeth and minimize decalcification. The ion exchange coating layer 110 can be used and reused. The regeneration reaction for the ion exchange coating 110 is reversible so that the ion exchange coating 110 is not permanently changed.

In some embodiments, the ion exchange coating layer 110 is substantially transparent to visible light of about 400 nm to about 750 nm when applied at a thickness of about 1 nm to about 200 nm on a substantially transparent shell 102. In various embodiments, the visible light transmission through the combined thickness of the shell 102 and the layer 110 is at least about 50%, or about 75%, or about 85%, or about 90%, or about 95%.

In some embodiments, the ion exchange coating layer 110 can include dyes or pigments to provide a desired color that may be, for example, decorative or selected to improve the appearance of the teeth of the patient.

The ion exchange coating layer 110 includes an ion exchange functional group that supply cations and/or anions to the surface of the tooth. In some examples, ion exchange functional groups that supply cations at the surface of the tooth can be strong acid resins (e.g., containing sulfonic acid groups (RSO₃H) such as sodium polystyrene sulfonate or poly(2-acrylamido-2-methyl-1-propanesulfonic acid) (polyAMPS) or weak acid resins (e.g., containing carboxylic acid groups (RCOOH)). Cationic exchange resins can exchange cationic (M⁺ⁿ) species including protons (H⁺) and metal ions. For example, the ion exchange coating layer 110 can be pre-charged and/or recharged with the cationic exchange resin having Ca⁺². Ions can be released into the proximity of the tooth in the presence of an alternative or different cationic species (M₊ₙ) species including protons (H⁺) and metal ions other than Ca⁺². Upon partial or full depletion of the Ca⁺² ions, the ion exchange coating layer 110 can be recharged with a Ca⁺² source that would displace the other (M⁺ⁿ) species including protons (H) and metal ions.

Similarly, with anionic resins, the ion exchange coating layer 110 can reversibly release anions (X^{m-}) to minimize demineralization. The anions can include hydroxide (HO⁻), fluoride (F⁻) and various phosphate ions (PO₄^{m-}) (including PO₄³⁻ and PO₄²⁻) and can be reversibly displaced in an oral environment with various anions. In some examples, ion exchange functional groups that supply anions at the surface of the tooth can be amines, such as strong-base exchangers as quaternary amines (RN(CH₃)₃⁺OH⁻) (e.g., trimethylammonium groups such as poly (acrylamido-N-propyltrimethylammonium chloride)) (polyAPTAC) and such as weak-base exchangers containing primary, secondary, and/or tertiary amines (e.g., polyethylene amine).

For a weak acid cation exchange resin, the ion exchange coating layer 110 can be based primarily on an acrylic or methacrylic acid that has been crosslinked with a di-functional monomer (e.g., divinylbenzene (DVB)). A method of manufacturing can include beginning with an ester of the acid in suspension polymerization coming before hydrolysis to create the functional acid group. The layer 110 with a weak acid cation can have an affinity for hydrogen ions and can be regenerated with strong acids. For the layer 110 with a strong acid cation, the layer 110 can include sulfonated copolymers of styrene and DVB, which can exchange cations or split neutral salts.

For an ion exchange coating layer 110 with a weak base resin, the layer 110 can have mechanical and chemical stability. For strong base resins, there can be two classes: Type 1 and Type 2. The Type 1 functional group can be a quaternized amine product made by the reaction of trimethylamine with the copolymer after chloromethylation. The regeneration efficiency of a Type 2 resin can be greater than a Type 1 resin. Type 2 functionality can be obtained by the reaction of the styrene-DVB copolymer with dimethylethanolamine.

In various embodiments, which are not intended to be limiting, the ion exchange coating layer 110 can supply ions in the mouth of the patient such as calcium, fluoride, phosphate and mixtures and combinations thereof. For example, in some embodiments, suitable calcium compounds supplied by the ion exchange coating layer 110 include calcium compounds including Ca²⁺ ions. Suitable calcium compounds include, but are not limited to, calcium chloride, calcium carbonate, calcium caseinate, calcium chloride, calcium citrate, calcium glubionate, calcium gluceptate, calcium glycerophosphate, calcium gluconate, calcium hydroxide, calcium hydroxyapatite, calcium lactate, calcium oxalate, calcium oxide, calcium pantothenate, calcium phosphate, calcium polycarbophil, calcium propionate, calcium pyrophosphate, calcium sulfate, and mixtures and combinations thereof. These compounds have been found to minimize demineralization of calcium hydroxyapatite at the surface of the tooth of a patient.

In some embodiments, the tooth re-mineralizing compounds supplied by the ion exchange coating layer 110 include phosphate compounds. Suitable phosphate compounds include, but are not limited to, aluminum phosphate, bone phosphate, calcium phosphate, calcium orthophosphate, calcium phosphate dibasic anhydrous, calcium phosphate-bone ash, calcium phosphate dibasic dihydrate, calcium phosphate dibasic anhydrous, calcium phosphate dibasic dihydrate, calcium phosphate tribasic, dibasic calcium phosphate dihydrate, dicalcium phosphate, neutral calcium phosphate, calcium orthophosphate, tricalcium phosphate, precipitated calcium phosphate, tertiary calcium phosphate, whitlockite, magnesium phosphate, potassium phosphate, dibasic potassium phosphate, dipotassium hydrogen orthophosphate, dipotassium monophosphate, dipotassium phosphate, monobasic potassium phosphate, potassium acid phosphate, potassium biphosphate, potassium dihydrogen orthophosphate, potassium hydrogen phosphate, sodium phosphate, anhydrous sodium phosphate, dibasic sodium phosphate, disodium hydrogen orthophosphate, disodium hydrogen orthophosphate dodecahydrate, disodium hydrogen phosphate, disodium phosphate, and mixtures and combinations thereof.

Fluoride compounds incorporated into the mineral surface of a tooth help inhibit the demineralization of enamel and protect the tooth. Fluoride compounds absorbed into mineral surfaces of a tooth attract calcium and phosphate ions from saliva, or other sources, which results in the formation of fluorapatite and protects the tooth against demineralization. While not wishing to be bound by any theory, currently available evidence indicates that fluorapatite exhibits lower solubility than naturally occurring hydroxyapatite, which can help resist the inevitable acid challenge that teeth face daily.

The ion exchange coating layer 110 includes a polymer with an ion exchange functional group covalently bonded thereto. The ion exchange functional group supplies in the oral environment in the mouth of a patient a controlled release of at least one therapeutically beneficial ion at a surface of a tooth. The ion exchange functional group in the layer 110 can include, but is not limited to, carboxylate, phosphate, phosphonate, sulfate, sulfonate, and mixtures and combinations thereof.

In some embodiments, which are not intended to be limiting, the polymer in the ion exchange coating layer 110 is a sulfopolyester or a polyester with a backbone having an aromatic nucleus with a metal sulfonate group RSO₃₋ attached thereto, wherein R is a functional group chosen from hydroxy, carboxy, amino, and combinations thereof. The metal sulfonate group can include a monovalent metal ion that is chosen from Na₊, Li⁺, K⁺, NH₄⁺, Ag⁺, and mixtures and combinations thereof.

Sulfopolyesters can spontaneously form small negatively charged aggregates suspended in an aqueous environment and are particularly well suited for the ion exchange coating layer 110. Suitable sulfopolyesters include repeat units from a dicarboxylic acid, a difunctional sulfomonomer, and a diol.

Suitable dicarboxylic acids for the sulfopolyester include, but are not limited to, naphthalene dicarboxylic acid or naphthalene dicarboxylate esters such as naphthalene-2,6-dicarboxylic acid. The naphthalene dicarboxylate monomer may be in the form of the free-acid or esterified derivatives thereof. High T_{g} (glass transition temperature) polyester resins are readily obtained when each of the aromatic rings bears one of the carboxyl(ate) groups.

The difunctional sulfomonomer component of the polyester may be a dicarboxylic acid or an ester thereof containing a metal sulfonate group (—SO₃₋), a diol containing a metal sulfonate group, or a hydroxy acid containing a metal sulfonate group. In some embodiments, the metal in the metal sulfonate group of the layer 110 can include, but is not limited to, Na⁺, Li⁺, K⁺, Mg⁺⁺, Ca²⁺, Ni²⁺, Fe²⁺, Fe³⁺, Zn²⁺, Sr²⁺, Ag⁺, Sn²⁺, an ammonium substituted with an alkyl or hydroxy alkyl radical having 1 to 4 carbon atoms, and combinations thereof. The metal can also be chosen from divalent alkali metal ions. The difunctional sulfomonomer component of the polyester may be a dicarboxylic acid or an ester thereof containing a metal sulfonate group (RSO₃⁻), a diol containing a metal sulfonate group, or a hydroxy acid containing a metal sulfonate group.

The difunctional sulfomonomer contains at least one sulfonate group attached to an aromatic nucleus wherein the functional groups are hydroxy, carboxy or amino. Advantageous difunctional sulfomonomer components are those wherein the sulfonate salt group is attached to an aromatic acid nucleus such as benzene, naphthalene, diphenyl, oxydiphenyl, sulfonyldiphenyl or methylenediphenyl nucleus. Examples of sulfomonomers include sulfophthalic acid, sulfoterephthalic acid, sulfoisophthalic acid, 5-sodiosulfoisophthalic acid, 4-sulfonaphthalene-2,7-dicarboxylic acid, and their esters. Metallosulfoaryl sulfonate may also be used as a sulfomonomer.

The sulfomonomer is present in an amount to provide water-dispersibility to the sulfopolyester. In some examples, the sulfomonomer is present in an amount of about 5 to about 40 mole percent, about 15 to about 25 mole percent, an amount greater than about 5 mole percent, or an amount less than about 50 mole percent based on the sum of the moles of total dicarboxylic acid content.

The diol component of the polyester consists of at least 35 mole percent of a diol selected from ethylene glycol, diethylene glycol, propane-1,2-diol, 1,4-cyclohexanedimethanol and 2,2-dimethyl-1,3-propanediol. The diol component may also include mixtures of the above diols. In addition, the diol component may include up to 65 mole percent of other cycloaliphatic diols preferably having 6 to 20 carbon atoms or aliphatic diols preferably having 3 to 20 carbon atoms. Included within the class of aliphatic diols are aliphatic diols having ether linkages such as polydiols having 4 to 800 carbon atoms. Examples of additional diols are: diethylene glycol, triethylene glycol, propane-1,3-diol, butane-1,4-diol, pentane-1,5-diol, hexane-1,6-diol, 3-methylpentanediol-(2,4), 2-methylpentanediol-(1,4), 2,2,4-trimethylpentane-diol-(1,3), 2-ethylhexanediol-(1,3), 2,2-diethylpropane-diol-(1,3), hexanediol-(1,3), 1,4-di-(hydroxyethoxy)-benzene, 2,2-bis-(4-hydroxycyclohexyl)-propane, 2,4-dihydroxy-1,1,3,3-tetramethyl-cyclobutane, 2,2-bis-(3-hydroxyethoxyphenyl)-propane, and 2,2-bis-(4-hydroxypropoxyphenyl)-propane. The diol component of the polyester may contain at least 95 mole percent of a diol selected from ethylene glycol, propane-1,2-diol, propane-1,3-diol, 1, 4-cyclohexanedimethanol and 2,2-dimethyl-1,3-propanediol.

The sulfopolyesters can be prepared by conventional polycondensation procedures well-known in the art. In some embodiments, sulfopolyesters are prepared by polymerizing glycols and aromatic diacids. Specifically, some of the monomers used to produce water-dispersible sulfopolyesters include isophthalic acid (IPA), 5-sodiosulfoisophthalic acid (5-SSIPA), 1,4-cyclohexanedimethanol (CHDM), and diethylene glycol (DEG).

The choice of cation can influence the properties of the resulting sulfopolyester. It is possible to prepare the sulfopolyester using, for example, a sodium sulfonate salt and later by ion exchange replace this ion with a different ion, for example, calcium, and thus alter the characteristics of the polymer. In general, this procedure may be preferred to preparing the polymer with divalent salts, as the sodium salts are usually more soluble in the polymer manufacturing components than are the divalent metal salts. Polymers containing divalent and trivalent metal ions are normally less elastic and rubber-like than polymers containing monovalent ions.

Cationic and anionic polymers can be characterized by their charge density, usually expressed as milliequivalents (meq) of anionic or cationic groups per gram of polymer. Calculated charge densities of water-dispersible sulfopolyesters can range from about 0.3 to about 0.9 meq/g (see Table 1 below). Sulfopolyesters with high charge density can disperse in water and form smaller aggregates in dispersions. Sulfopolyesters with low charge density can produce films with increased water/humidity resistance.

Some water-dispersible sulfopolyesters can have number-average molecular weight M. of about 5 kDa to about 15 kDa, weight-average molecular weight M_{W}=20-30 kDa, and polydispersity index M_{W}/Mₙ of about 2. Sulfopolyesters can span a range of T_{g} from about 0° C. to about 65° C. (see Table 1).

Suitable sulfopolyesters for the ion exchange coating layer 110 include, but are not limited to, those available from Eastman Chemical, Kingsport, Tenn., under the trade designations EASTMAN AQ and EASTEK (see Table 1 below).

TABLE 1
Typical properties of Eastman water-dispersible sulfopolyesters

| | | | Calculated |
|---|---|---|---|
| | | Approximate | charge |
| | | T_{g} | density |
| Solid Form | Dispersion Form | (° C.) | (meq/g) |
| Eastman AQ 1350 | — | 0 | 0.32 |
| copolyester | | | |
| Eastman AQ 1950 | — | 3 | 0.38 |
| copolyester | | | |
| Eastman AQ 2350 | — | 10 | 0.38 |
| copolyester | | | |
| — | Eastek 1400 polymer | 29 | 0.45 |
| | disperson | | |
| — | Eastek 1300 polymer | 35 | 0.47 |
| | disperson | | |
| Eastman AQ 385 | Eastek 1000 polymer | 38 | 0.43 |
| polymer | disperson | | |
| Eastman AQ 48 ultra | — | 48 | 0.89 |
| polymer | | | |
| Eastman AQ 555 | Eastek 1100 polymer | 55 | 0.66 |
| polymer | disperson | | |
| Eastman AQ 655 | Eastek 1200 polymer | 63 | 0.33 |
| polymer | disperson | | |

The flexibility of sulfopolyester can be modified with a variety of plasticizers, e.g., propylene glycol, glycerin, triethyl citrate (TEC), and 2-butoxyethanol. Depending on the polymeric materials selected for the shell 102, the end use of the dental appliance, and the needs of the patient, more or less flexibility may be needed.

In some embodiments, more than one sulfopolyester may be present in the ion exchange coating layer 110 to provide desired properties of the layer or to maintain effective ion exchange, such as calcium ion uptake.

In another embodiment, the ion exchange functional group of the layer 110 can include a quaternary ammonium compound chosen from tetraalkylammoniums, alkylated pyridines, alkylated immidazoles, phosphonium and combinations thereof. These ion exchange functional groups, including quaternary ammonium compounds, can be tethered to carbon backbones to help retain counter ions, e.g., fluoride. In some embodiments, the ion exchange functional group of the layer 110 is chosen from carboxylate, phosphate, phosphonate, sulfate, sulfonate and combinations thereof, and quaternary ammonium, imidazolium, pyridinium, phosphonium and combinations thereof.

The shell 102 of the orthodontic appliance 100 is an elastic polymeric material that generally conforms to a patient's teeth, and may be transparent, translucent, or opaque. In some embodiments, the shell 102 is a clear or substantially transparent polymeric material that may include, for example, one or more of amorphous thermoplastic polymers, semi-crystalline thermoplastic polymers and transparent thermoplastic polymers chosen from polycarbonate, thermoplastic polyurethane, acrylic, polysulfone, polypropylene, polypropylene/ethylene copolymer, cyclic olefin polymer/copolymer, poly-4-methyl-1-pentene or polyester/polycarbonate copolymer, styrenic polymeric materials, polyamide, polymethylpentene, polyetheretherketone and combinations thereof. In another embodiment, the shell 102 may be chosen from clear or substantially transparent semi-crystalline thermoplastic, crystalline thermoplastics and composites, such as polyamide, polyethylene terephthalate, polybutylene terephthalate, polyester/polycarbonate copolymer, polyolefin, cyclic olefin polymer, styrenic copolymer, polyetherimide, polyetheretherketone, polyethersulfone, polytrimethylene terephthalate, parylene (poly(p-xylene), and mixtures and combinations thereof. In some embodiments, the shell 102 may partially comprise parylene, substantially comprise parylene or may be comprised essentially entirely of parylene, as described in U.S. Provisional Patent Applications Ser. No. 62/736,774 and PCT Patent Application No. US2018/043380, which are incorporated by reference in their entirety.

In some embodiments, the shell 102 is a polymeric material chosen from polyethylene terephthalate, polyethylene terephthalate glycol, poly cyclohexylenedimethylene terephthalate glycol, and mixtures and combinations thereof. One example of a commercially available material suitable as the elastic polymeric material for the shell 102, which is not intended to be limiting, is PETg. Suitable PETg resins can be obtained from various commercial suppliers such as, for example, Eastman Chemical, Kingsport, Tenn.; SK Chemicals, Irvine, Calif.; DowDuPont, Midland, Mich.; Pacur, Oshkosh, Wis.; and Scheu Dental Tech, Iserlohn, Germany.

In some embodiments, the shell 102 may be made of a single polymeric material or may include multiple layers of different polymeric materials.

In one embodiment, the shell 102 is a substantially transparent polymeric material. In this application the term substantially transparent refers to materials that pass light in the wavelength region sensitive to the human eye (about 400 nm to about 750 nm) while rejecting light in other regions of the electromagnetic spectrum. In some embodiments, the reflective edge of the polymeric material selected for the shell 102 should be above about 750 nm, just out of the sensitivity of the human eye.

The ion exchange coating layer 110 can be formed on the surfaces 106, 108 of the shell 102 by any suitable coating technique. In one non-limiting embodiment, the ion exchange coating layer is applied on a substantially flat polymeric film with a Mayer rod, and the polymeric film is formed into a dental appliance. Other techniques for applying the ion exchange coating layer 100 include, for example, vapor deposition, sputtering, spraying, dipping, and the like, either on a flat film that is subsequently thermoformed into a dental appliance, or on a previously formed dental appliance.

The ion exchange coating layer 110 can be formed as a thin film on the polymeric substrate, and the thin film should have a thickness no greater than needed to provide release of metal ions on a sustainable basis over a suitable period of time in the mouth of the patient. In various embodiments, for example, the thickness of the ion exchange coating layer 110 will vary at least in part depending on the metals selected for the coating (which can impact, for example, the solubility and abrasion resistance of the coating layer). In various embodiments, the ion exchange coating layer 110 should be sufficiently thin such that the layer 110 does not interfere with the dimensional tolerances or flexibility of the shell 102. For example, suitable ion exchange coating layers have a thickness of about 1 micrometer (μm) to about 5 μm, or about 1 μm to about 3 μm, but thinner or thicker coatings may be used depending on, for example, the degree of ion release needed over a period of time.

The major surface of the polymeric sheet to which the layer of the ion exchange coating is applied may optionally be chemically or mechanically treated prior to applying the layer of the ion exchange coating to, for example, enhance adhesion between the layer and the substrate.

A plurality of cavities may then be formed in the sheet of polymeric material to form a dental appliance, wherein the cavities are configured to receive one or more teeth. The cavities may be formed by any suitable technique, including thermoforming, laser processing, chemical or physical etching, and combinations thereof.

The applied ion exchange coating may be continuous or discontinuous on the side of the formed dental appliance, and in some embodiments the coverage in the tooth-like cavities of the shell should be greater than about 70%, greater than 80%, greater than 90%, or greater than 95%, to provide an effective amount of ions at the surface of the tooth. In some embodiments, the ion exchange coating is present in fully continuous layer providing 100% coverage in the tooth-like cavities of the shell. In some embodiments, the dimension of the surface area of any discontinuous or discrete coating in either direction is greater than 100 nm, which ensures that the discontinuous or discrete ion exchange coating is bound very well to the surface of the polymeric substrate.

In another embodiment, the tooth-shaped cavities may be formed in the sheet of polymeric material to form a shell-like orthodontic dental appliance, and then the layer of the ion exchange coating may thereafter be applied to overlie all or a desired portion of the cavities. In some embodiments, the layer of the ion exchange coating may also be applied on all or a desired portion of an external surface of the dental appliance opposite the teeth-retaining cavities.

In another embodiment, the shell-like dental appliance may be formed using a three-dimensional (3D) printing process (e.g., additive manufacturing), such as stereolithography, and then the ion exchange coating layer may thereafter be applied on an internal surface of the tooth-retaining cavities, or on an external surface, or both.

In some example embodiments, the dental appliance may be made by applying a first coating composition including at least one polymer with an ion exchange functional group covalently bonded thereto on at least one major surface of a substantially flat sheet of a polymeric material. In various embodiments, the first coating composition further includes water and an optional surfactant.

Suitable surfactants include but are not limited to conventional anionic, cationic and/or non-ionic surfactants such as Na, K and NH₄ salts of dialkylsulphosuccinates, Na, K and NH₄ salts of sulphated oils, Na, K and NH₄ salts of alkyl sulphonic acids, Na, K and NH₄ alkyl sulphates, alkali metal salts of sulphonic acids, fatty alcohols, ethoxylated fatty acids and/or fatty amides, and Na, K and NH₄ salts of fatty acids such as Na stearate and Na oleate. Some anionic surfactants include alkyl or (alk)aryl groups linked to sulphonic acid groups, sulphuric acid half ester groups (linked in turn to polyglycol ether groups), phosphonic acid groups, phosphoric acid analogues and phosphates or carboxylic acid groups. Cationic surfactants include alkyl or (alk)aryl groups linked to quaternary ammonium salt groups. Non-ionic surfactants include polyglycol ether compounds and polyethylene oxide compounds. In some example embodiments examples of surfactants may include sodium bis(tridecyl) sulfosuccinnate, di(2-ethylhexyl) sodium sulfosuccinnate, sodium dihexylsulfosuccinnate, sodium dicyclohexyl sulfosuccinnate, diamyl sodium sulfosuccinnate, sodium diisobutyl sulfosuccinate, disodium isodecyl sulfosuccinnate, disodium ethoxylated alcohol half ester of sulfosuccinnic acid, disodium alkyl amido polyethoxy sulfosuccinnate, tetrasodium N-(1,2-dicarboxy-ethyl)-N-oxtadecyl sulfosuccinnamate, disodium N-octasulfosuccinnamate, sulfated ethoxylated nonylphenol, 2-amino-2-methyl-1-propanol, and the like.

The first coating composition can be dried to form a first coating prior to thermally forming tooth-retaining cavities in the flat sheet of polymeric material.

A second coating composition including a second therapeutically beneficial metal ion, different from the first metal ion, is then applied to the first coating on the thermally formed polymeric shell. The second coating composition further includes water and an optional surfactant.

The first metal ion in the first coating is then substantially replaced with the therapeutically beneficial second metal ion to form a second therapeutic coating on the polymeric shell, which can then optionally be dried. The second therapeutic coating can supply in an oral environment a controlled release of at least one of the therapeutically beneficial ions at the surface of a tooth.

In some example embodiments, which are not intended to be limiting, the therapeutically beneficial second metal ion is a divalent alkali metal cation, and can be chosen from Mg²⁺, Ca²⁺, Ni²⁺, Fe²⁺, Sr²⁺, Sn²⁺, and mixtures and combinations thereof. In additional embodiments, the second metal ion is a metal anion chosen from, for example, fluoride, phosphate, and combinations thereof.

The second coating composition may include any ionic solution capable of exchanging the second therapeutically beneficial ion for the first ion to form the second coating composition, and non-limiting examples of suitable second coating compositions capable of exchanging Ca²⁺ ions include CaCl₂, Ca(NO₃)₂, calcium gluconate, calcium gluconate lactate, and mixtures and combinations thereof.

The thickness of the second therapeutic coating can vary widely depending on the intended application, and suitable examples have a thickness greater than about 1 micron, between about 1 micron to about 10 microns, and between about 1 micron and about 5 microns.

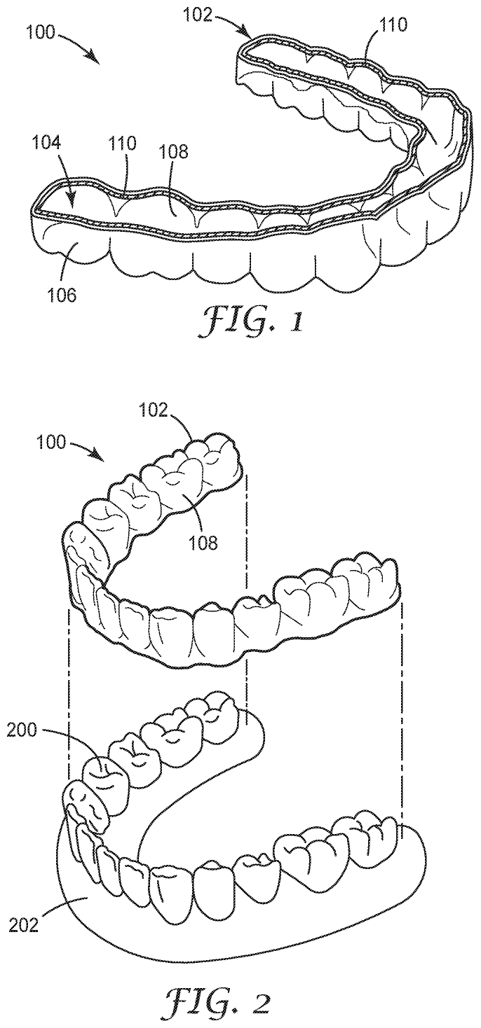

Referring now to FIG. 2, in one example embodiment the shell 102 of the dental appliance 100 is an elastic polymeric material that generally conforms to a patient's teeth 200, but that is slightly out of alignment with the patient's initial tooth configuration. In some embodiments, the shell 102 may be one of a group or a series of shells having substantially the same shape or mold, but which are formed from different materials to provide a different stiffness or resilience as need to move the teeth of the patient. In this manner, in one embodiment, a patient or a user may alternately use one of the orthodontic appliances during each treatment stage depending upon the patient's preferred usage time or desired treatment time period for each treatment stage.

In one example, FIG. 2 may represent the shell 102 of the dental appliance 100, which is an elastic polymeric material, generally conforms to a patient's teeth for the purpose of maintaining tooth position, as in the case of an orthodontic retainer. FIG. 2 may also represent the shell 102 of the dental appliance 100, wherein one of the primary purposes of the dental appliance is to provide the controlled release of at least one therapeutically beneficial ion at a surface of a tooth in an oral environment via the ion exchange coating.

No wires or other means may be provided for holding the shell 102 over the teeth 200, but in some embodiments, it may be desirable or necessary to provide individual anchors on teeth with corresponding receptacles or apertures in the shell 102 so that the shell 102 can apply a retentive or other directional orthodontic force on the tooth which would not be possible in the absence of such an anchor.

The shells 102 may be customized, for example, for day time use and night time use, during function or non-function (chewing vs. non-chewing), during social settings (where appearance may be more important) and nonsocial settings (where the aesthetic appearance may not be a significant factor), or based on the patient's desire to accelerate the teeth movement (by optionally using the more stiff appliance for a longer period of time as opposed to the less stiff appliance for each treatment stage).

For example, in one aspect, the patient may be provided with a clear dental appliance that may be primarily used to retain the position of the teeth, and an opaque dental appliance that may be primarily used to move the teeth for each treatment stage. Accordingly, during the day time, in social settings, or otherwise in an environment where the patient is more acutely aware of the physical appearance, the patient may use the clear appliance. Moreover, during the evening or night time, in non-social settings, or otherwise when in an environment where physical appearance is less important, the patient may use the opaque appliance that is configured to apply a different amount of force or otherwise has a stiffer configuration to accelerate the teeth movement during each treatment stage. This approach may be repeated so that each of the pair of appliances are alternately used during each treatment stage.

Referring again to FIG. 2, systems and methods include a plurality of incremental position adjustment appliances, each formed from the same or a different material, for each treatment stage of orthodontic treatment. The dental appliances may be configured to incrementally reposition individual teeth 200 in an upper or lower jaw 202 of a patient and the cavities 104 incrementally move one or more teeth. In some embodiments, the cavities 104 are configured such that selected teeth will be repositioned, while others of the teeth will be designated as a base or anchor region for holding the repositioning appliance in place as it applies the resilient repositioning force against the tooth or teeth intended to be repositioned.

Placement of the shell 102, which may be an elastic positioner, over the teeth 200 applies controlled forces in specific locations to gradually move the teeth into the new configuration. Repetition of this process with successive appliances having different configurations eventually moves a patient's teeth through a series of intermediate configurations to a final desired configuration.

In some embodiments, the dental appliance can be used in a method of treating demineralization of a surface of a tooth. The method can include positioning a dental appliance adjacent to the surface of the tooth, wherein the dental appliance has a polymeric shell with tooth-retaining cavities. The polymeric shell has a coating that has a polymer with an ion exchange functional group covalently bonded thereto, and the ion exchange functional group is configured to supply in an oral environment a controlled release of at least one therapeutically beneficial ion at a surface of a tooth. In some examples, the polymer can supply either therapeutically beneficial cations or anions, or both, at the surface of the tooth. For example, the therapeutically beneficial ion supplied at the surface of the tooth can be chosen from calcium, fluoride, phosphate and combinations thereof. In some examples, the method of treating demineralization of a surface of a tooth can include additional steps of incrementally moving one or more teeth from a maloccluded position to a desired position in the mouth of a patient.

In some embodiments, the polymeric shell includes a coating with an ion exchange functional group, and the ion exchange functional group includes a first metal ion. The coated polymeric shell can then be exposed to an ionic solution with a second dentally therapeutic metal ion, different from the first metal ion. The therapeutically beneficial ion supplied at the surface of the tooth can be chosen from, for example, calcium, fluoride, phosphate and combinations thereof. In some examples, the ionic solution can be an aqueous solution containing the ion, and in some embodiments the aqueous solution can about 1% to about 10% by weight of the therapeutically beneficial ion. For example, in some embodiments the ionic solution can include Ca²⁺ ions, and non-limiting examples of suitable ionic solutions include aqueous solutions of CaBr₂, CaCl₂, and the like.

In the ionic solution the first metal ion on at least a portion of the ion exchange functional group on the coating on the dental appliance is replaced with the second metal ion to form a second therapeutically beneficial coating. For example, the second therapeutically beneficial metal ion can be chosen from calcium, fluoride, phosphate, and combinations thereof. The dental appliance with the second therapeutically beneficial metal coating is then positioned in the mouth of the patient adjacent to a surface of at least one tooth. The second therapeutically beneficial coating supplies in an oral environment a controlled release of at least one therapeutically beneficial ion at the surface of the tooth.

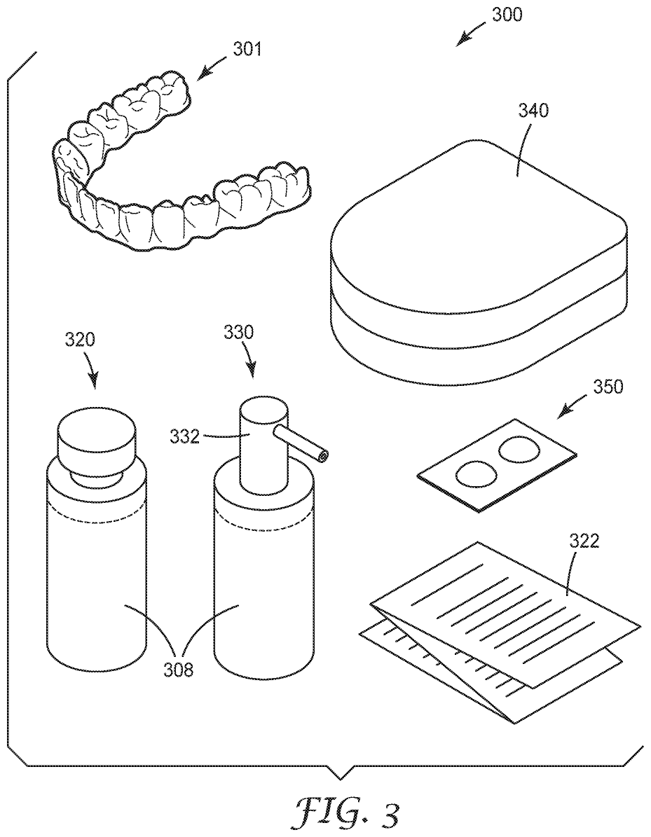

In some embodiments, the coating layer can be used in conjunction with a cleaning/replenishing solution containing an effective amount of therapeutically effective ions to help replenish the ions of the layer when the orthodontic appliance is not being worn about the teeth of the patient. Referring to FIG. 3, in various embodiments the system may be supplied in the form of a kit 300 including the dental appliance 301 with an ion exchange coating thereon and an aqueous ionic solution 308. In one embodiment, the aqueous ionic solution 308 may be supplied in a container 320 such as, for example, a squeezable bottle or a collapsible tube, along with instructions 322 for proper application to the dental appliance 301. In another embodiment, the aqueous ionic solution 308 may be supplied in a dispenser 330, wherein the dispenser 330 includes for example, a syringe, a trigger-operated gun, or a pump 332 configured to dispense a predetermined amount of the aqueous ionic solution 308 for each insertion of the dental appliance 301 into the mouth of the patient. In another embodiment, the dispenser 330 may be configured to automatically dispense a predetermined amount of the aqueous ionic solution 308 for each insertion of the dental appliance 301. In another embodiment, the kit may optionally include additional items such as, for example, a storage case 340, which may serve as a holder or an automated cleaning apparatus for temporary storage of the dental appliance 301 while not the dental appliance is not in the mouth of the patient, liquid cleaning or disinfecting solutions or solid tablets 350 dissolvable in water for use with the storage case or automated cleaning apparatus, a charger for the automated dispenser or cleaning apparatus, instructions for use, and the like.

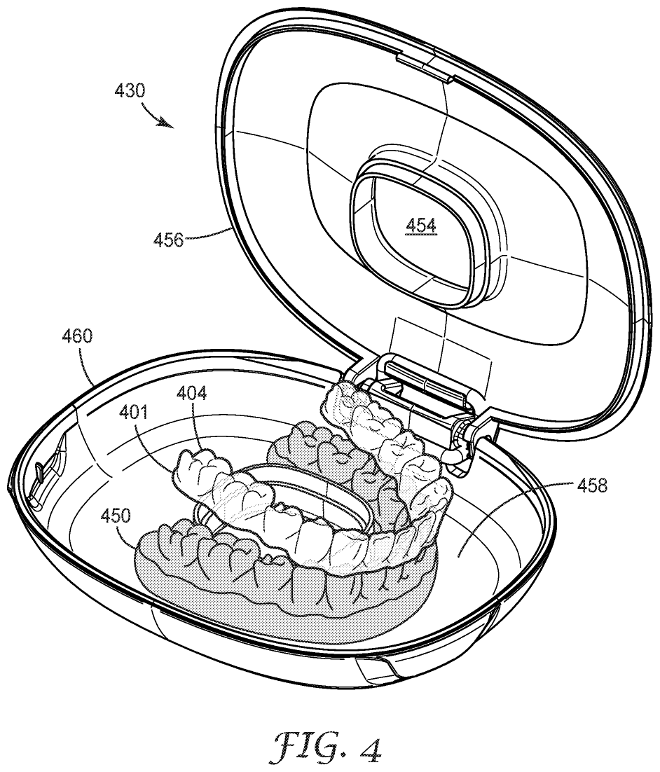

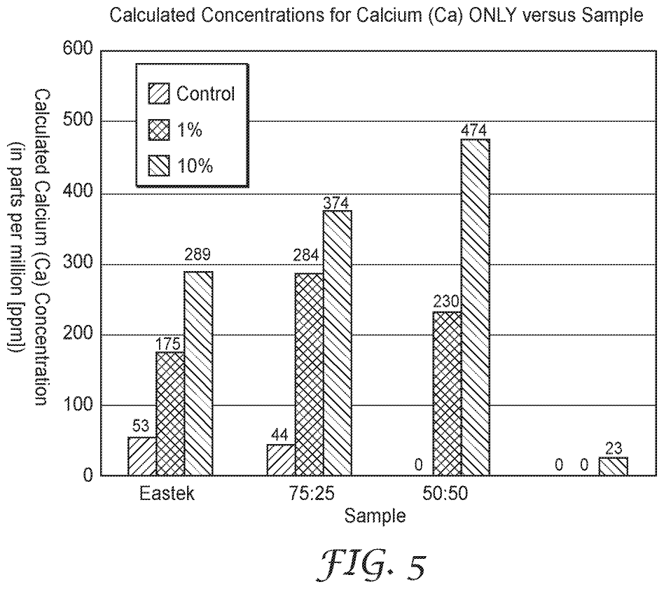

As shown in FIG. 4, in one example embodiment a re-closable storage unit and dispenser 430 includes a foam applicator pad 450 shaped to hold a shell-like dental appliance 401 including tooth-retaining cavities 404. When an aqueous ionic liquid (not shown in FIG. 4) is dispensed into an opening 454 in a hinged cover 456 of the dispenser 430, the aqueous ionic liquid collects in a reservoir 458 in a bottom portion 460 of the dispenser. The foam applicator pad 450 absorbs the aqueous ionic liquid from the reservoir 458. When the cover 456 is closed and engages the bottom portion 460 of the dispenser 430, the dental appliance 401 is pressed against the foam applicator pad 450. The foam applicator pad 450 dispenses a predetermined measured amount of the aqueous ionic liquid into the cavities 404 of the dental appliance 401.

The devices of the present disclosure will now be further described in the following non-limiting examples.

### Examples

In general, the process included:
1. PET sheet was coated with a sulfopolyester coating including sulfonic groups that acted as ion exchange sites;
2. the coated PET sheet was dipped in CaBr₂ solution, where the Na⁺ ions were replaced by Ca²⁺ ions;
3. Ca²⁺ counter ions were then part of the coated film; and
4. when introduced in DI water (or oral environment), Ca²⁺ was ion exchanged.

### Experimental

### Materials

Eastek 1000D was received from Eastman Chemical Company and used as received without further purification (available from Eastman Chemical. Kingsport, Tenn.). Dynol 607 was diluted to 10 wt % in water before use (Air Products and Chemicals, Inc., Allentown, Pa.). WB-50 sulfonated polyester was synthesized by Film Manufacturing Supply Chain Operations (FMSCO) of 3M Company (available from 3M Company, Maplewood, Minn.).

### Synthesis of WB-50—Water Soluble Sulfopolyester

To a clean, dry oil-jacketed 100-gallon stainless steel reactor, the following materials were added:

125.1 lbs (56.7 kg) of terephthalic acid (TA);

23.4 lbs (10.6 kg) of sodiosulfoisophthalic acid (SSIPA);

123.7 lbs (56.1 kg) of isophthalic acid (IPA);

123.7 lbs (56.1 kg) of neopentyl glycol (NPG);

146.8 lbs (66.6 kg) of ethylene glycol (EG);

126.6 g of antimony triacetate (AT); and

318.5 g of sodium acetate (SA)

The kettle was placed under 30 psig of nitrogen pressure. The contents of the vessel were heated, and a typical polyethylene terephthalate (PET) transesterification took place. The batch was heated to ˜485° F. (˜252° C.). Once esterification was determined to be complete, pressure in the kettle was slowly vented.

A typical polyester polymerization was commenced. Vacuum was slowly pulled on the kettle, and heat was applied. Excess glycol was removed. Eventually, the kettle reached a temperature of about 525° F. and a vacuum measuring as low as 1.5 mmHg. Once target initial viscosity (IV) of about 0.50 dL/g was achieved, the batch was pressurized (under nitrogen) and drained into trays. These trays of resin were ground up and utilized for WB-50 solution making. The resultant polymer was composed of about:

5.5 mol % SSIPA, 47.5 mol % TA and 47 mol % IPA (on an acids basis); and

75% mol % NPG and 25 mol % EG (on a diols basis).

### Dispersion of Sulfonated Polyester and Solution Preparation

Solid WB50 sulfonated polyester was dispersed in water following a typical manufacturing procedure, which was adapted to lab scale equipment. Solid polyester (20 g) was first added to a 250 mL two-neck round bottom flask. A water:IPA mixture (4:1 by mass, 100 g) was added to the flask. The flask was equipped with a thermocouple, which was submerged into the aqueous solution and a reflux condenser under nitrogen. The solution was stirred with a magnetic stir bar and heated to reflux (approximately 85° C.) using a heated oil bath. Once a homogeneous solution was formed, the solution was refluxed for one hour to ensure complete dispersion of the polyester. The reflux condenser was then removed and replaced with a glass short-path distillation head. The solution was then heated to 94° C., and isopropanol was collected in a round bottom flask (thermometer reading on distillation head read approximately 84° C.). The solution was held until the system stopped collecting distillate. The solution was then cooled to room temperature and filtered through a 120 mesh stainless steel screen to remove small precipitates. WB50 solution was then mixed with Eastek 1000D at varying ratios and two drops of Dynol solution per 20 mL sulfonated polyester solution were added.

### Solution Coating of Polyester Film

Aqueous solutions of sulfonated polyester were coated onto 3-millimeter sulfonated polyester primed PET using an RS06 Mayer rod. Coatings were dried at 90° C. in a batch oven for 5 minutes to ensure removal of water. The coating thickness is estimated to be about 3 μm thick.

### Ion Exchange

Solutions of calcium bromide in DI water at various weight fractions (1% and 10%) were prepared and 2 inch (5 cm) wide strips of coated PET (and an uncoated control) were submerged in the solution. Films were soaked for 48 hours to allow for equilibrium of ion exchange. After ion exchange, films were removed and rinsed with DI water and dried with a tissue cleaner (e.g., Kimwipes available from Kimberly-Clark, Roswell, Ga.) to remove excess solution from the surface.

### XRF Sample Preparation

Prior to data collection, one (1) aliquot was cut from each as-received sample using a Qualitest APC-3000 auto-pneumatic clicker press (available from APC. West Kingston, R.I.) and a circular specimen cutter 37 mm in diameter. Each obtained aliquot was then placed into a stainless steel X-ray fluorescence (XRF) sample bolder, secured into position using an aluminum hollow cavity mount, and analyzed for most of the elements in the periodic table [from carbon (C) to uranium (U) inclusive] using a Rigaku Primus II wavelength dispersive X-ray fluorescence spectrometer (available from Rigaku, Tokyo, Japan) equipped with a rhodium (Rh) X-ray source, a vacuum atmosphere, and a 20 mm diameter measurement area. Each aliquot was analyzed three times and an average and standard deviation calculated and report for each element detected.

### Results

### Calcium Content

Calcium ion exchange efficiency was measured using XRF. A measurable amount of calcium ions were present in coated film samples, and statistically significant differences were observed between samples prior to exposure to solution and those submerged in differing concentrations as shown in Table 2. Furthermore, uncoated PET samples did not show a significant calcium composition after soaking in the aqueous solution.

### SUMMARY

The calculated calcium (Ca) concentrations, obtained by semi-quantitative XRF analysis and reported in parts per million (ppm), are listed in Table 2. Based on the results, the following were noted:
1. Calcium (Ca) was detected in all of the samples, except the 50:50 control sample, the PET control sample, and the PET 1% sample.
2. For both the Eastek sample set and the 75:25 sample set only, the calcium (Ca) amount was highest in the 10% sample followed by, in decreasing amounts of calcium (Ca), the 1% sample and the control sample respectively. Note that the calcium (Ca) levels in the control samples were significantly lower in comparison to the respective 1% and 10% samples for both the Eastek and 75:25 sample sets.
3. For the 50:50 sample set only, the calcium (Ca) amount was highest in the 10% sample followed by, in decreasing amount of calcium (Ca), the 1% sample. No calcium (Ca) was detected in the control sample.
4. For the PET sample set only, calcium (Ca) was detected only in the 10% sample.
5. For the 1% samples only within each sample set, note that the calcium (Ca) amount increased from the Eastek sample to the 75:25 sample, but decreased from the 75:25 sample to the 50:50 sample. No calcium (Ca) was detected in the PET sample.
6. For the 10% samples only within each sample set, note that the calcium (Ca) amount increased from the Eastek sample to the 75:25 sample to the 50:50 sample, but significantly decreased from the 50:50 sample to the PET sample. Comparing the 10% sample only in the Eastek, the 75:25, and the 50:50 sample sets, the amount of calcium (Ca) was increasing with decreasing Eastek 1000 percentage (or increasing with increasing WB50 percentage).

Other elements, especially carbon (C) and oxygen (O) along with sodium (Na) and sulfur (S) were also detected in the majority of the samples.

### Sample Description

Eastek is PET film coated with Eastek 1000; 75:25 is PET film coated with blend of 75% Eastek 1000/25% WB50; 50:50 is PET film coated with blend of 50% Eastek 1000/50% WB50; and PET is uncoated PET film.

TABLE 2
Calculated Concentrations (ppm) for
Calcium (Ca) ONLY versus Sample

| | Sample | | | |
|---|---|---|---|---|
| Solutions of calcium | Eastek | 75%:25% | 50%:50% | PET |
| bromide in DI water | (ppm Ca) | (ppm Ca) | (ppm Ca) | (ppm Ca) |
| Control | 53 | 44 | 0 | 0 |
| 1% | 175 | 284 | 230 | 0 |
| 10% | 289 | 374 | 474 | 23 |

Furthermore, from Tables 3-5 it appears that sodium ions were displaced during the ion exchange.

TABLE 3
Composition of sample prior to soaking in aqueous solution

| | Calculated Semi-Quantitative XRF Results | | | | | | | |
|---|---|---|---|---|---|---|---|---|
| | (reported in mass percent [%]) | | | | | | | |
| | Control Samples ONLY | | | | | | | |
| Element | Eastek | | 75%:25% | | 50%:50% | | PET | |
| (Symbol) | Avg. | Dev. | Avg. | Dev. | Avg. | Dev. | Avg. | Dev. |
| Carbon (C) | 58.2 | 0.2 | 58.7 | 0.2 | 59.7 | 0.3 | 60.7 | 0.2 |
| Oxygen (O) | 40.6 | 0.2 | 40.2 | 0.2 | 39.5 | 0.3 | 39.3 | 0.2 |
| Sodium (Na) | 0.964 | 0.018 | 0.859 | 0.006 | 0.631 | 0.011 | ND | ND |
| Sulfur (S) | 0.265 | 0.004 | 0.229 | 0.001 | 0.154 | 0 | ND | ND |

“ND” = Not Detected;
“Avg.” = Average; and
“Dev.” = Standard Deviation

TABLE 4
Composition of sample after soaking in 1% CaBr₂ solution

| | Calculated Semi-Quantitative XRF Results | | | | | | | |
|---|---|---|---|---|---|---|---|---|
| | (reported in mass percent [%]) | | | | | | | |
| | 1% Samples ONLY | | | | | | | |
| Element | Eastek | | 75%:25% | | 50%:50% | | PET | |
| (Symbol) | Avg. | Dev. | Avg. | Dev. | Avg. | Dev. | Avg. | Dev. |
| Carbon (C) | 60 | 0.1 | 59.8 | 0.3 | 60.4 | 0.3 | 60.9 | 0.1 |
| Oxygen (O) | 39.8 | 0.1 | 39.9 | 0.3 | 39.4 | 0.3 | 39.1 | 0.1 |
| Sodium (Na) | 0.0113 | 0.0015 | 0.0145 | 0.0022 | 0.0087 | 0.0009 | ND | ND |
| Sulfur (S) | 0.114 | 0.002 | 0.179 | 0.002 | 0.156 | 0.002 | ND | ND |

“ND” = Not Detected;
“Avg.” = Average; and
“Dev.” = Standard Deviation

TABLE 5
Composition of sample after soaking in 10% CaBr₂ solution

| | Calculated Semi-Quantitative XRF Results | | | | | | | |
|---|---|---|---|---|---|---|---|---|
| | (reported in mass percent [%]) | | | | | | | |
| | 1% Samples ONLY | | | | | | | |
| Element | Eastek | | 75%:25% | | 50%:50% | | PET | |
| (Symbol) | Avg. | Dev. | Avg. | Dev. | Avg. | Dev. | Avg. | Dev. |
| Carbon (C) | 59.8 | 0.5 | 60.2 | 0.2 | 60.5 | 0.2 | 60.7 | 0.2 |
| Oxygen (O) | 40.0 | 0.5 | 39.5 | 0.2 | 39.3 | 0.2 | 39.3 | 0.2 |
| Sodium (Na) | ND | ND | 0.0360 | 0.0054 | 0.0192 | 0.0027 | ND | ND |
| Sulfur (S) | 0.192 | 0.007 | 0.196 | 0.001 | 0.145 | 0.003 | ND | ND |

“ND” = Not Detected;
“Avg.” = Average; and
“Dev.” = Standard Deviation

One aliquot was obtained from each as-received submitted sample and each aliquot was analyzed three times. The semi-quantitative results (reported in mass percent [%]) are listed in Tables 2-5 and reflect the average analyte concentrations in the aliquots obtained. The reported uncertainties are one standard deviation of the three measurements, rounded to provide at least three significant figures for the average concentrations with the deviation rounded to the same decimal place as the corresponding average. Elements designated by “Not Detected” in the results tabulated in the data section were either below the detection limit or not within the carbon (C) to uranium (U) elemental range utilized by the XRF instrument.

The SQX program (provided by Rigaku) for semi-quantitative XRF analysis divides individual elemental intensity data by the total intensity observed for the sample, accounting for absorption/enhancement effects using fundamental parameter algorithms and a developed sensitivity library. The results were normalized to 100% within the elemental range utilized (in this case, from carbon [C] to uranium [U] only). If the samples contain elements not included in this normalization, the elemental concentrations for the reported elements could be higher than the true values.

The results obtained via SQX may be useful for relative comparisons among samples but should be used with caution for absolute determinations of elemental concentrations. Full quantitative analysis may require either matrix-matched XRF standards or another elemental analysis technique, such as Inductively Coupled Plasma (ICP).

Various embodiments of the invention have been described. These and other embodiments are within the scope of the following claims.

### Embodiment A

A dental appliance comprising:

a polymeric shell with an arrangement of cavities configured to receive one or more teeth; and
a coating on at least portion of the polymeric shell, wherein the coating comprises a polyester with an ion exchange functional group covalently bonded thereto, and wherein the ion exchange functional group releases in an oral environment at least one therapeutically beneficial ion at a surface of a tooth.

### Embodiment B

The dental appliance of embodiment A, wherein the ion exchange functional group supplies cations at the surface of the tooth.

### Embodiment C

The dental appliance of embodiment A or B, wherein the ion exchange functional group supplies anions at the surface of the tooth.

### Embodiment D

The dental appliance as in one of embodiments A-C, wherein the ion supplied at the surface of the tooth is chosen from calcium, fluoride, phosphate and combinations thereof.

### Embodiment E

The dental appliance as in one of embodiments A-D, wherein the ion exchange functional group is chosen from carboxylate, phosphate, phosphonate, sulfate, sulfonate and combinations thereof.

### Embodiment F

The dental appliance as in one of embodiments A, B, D and E, wherein the polyester comprises a sulfopolyester.

### Embodiment G

The dental appliance as in one of embodiments A-F, wherein the polyester comprises a backbone having an aromatic nucleus with a metal sulfonate group RSO₃₋ attached thereto, and wherein R is a functional group chosen from hydroxy, carboxy, amino, and combinations thereof.

### Embodiment H

The dental appliance as in one of embodiments A-G, wherein the metal in the metal sulfonate group is chosen from Na⁺, Li⁺, K, Mg⁺⁺, Ca²⁺, Ni²⁺, Fe²⁺, Fe³⁺, Zn²⁺, Sr²⁺, Ag⁺, Sn²⁺, an ammonium substituted with an alkyl or hydroxy alkyl radical having 1 to 4 carbon atoms, and combinations thereof.

### Embodiment I

The dental appliance as in embodiment H, wherein the metal is chosen from divalent alkali metal ions.

### Embodiment J

The dental appliance as in embodiment I, wherein the metal ion is Ca².

### Embodiment K

The dental appliance as in one of embodiments A-J, wherein the ion exchange functional group comprise a quaternary ammonium compound chosen from tetraalkylammoniums, alkylated pyridines, alkylated immidazoles, phosphonium and combinations thereof.

### Embodiment L

The dental appliance as in one of embodiments A-K, wherein the ion exchange functional group is chosen from carboxylate, phosphate, phosphonate, sulfate, sulfonate and combinations thereof, and quaternary ammonium, imidazolium, pyridinium, phosphonium and combinations thereof.

### Embodiment M

The dental appliance as in one of embodiment A-L, wherein the ion supplied from the ion exchange compound to the surface of the tooth is F.

### Embodiment N

The dental appliance as in one of embodiments A-M, wherein the cavities are configured to incrementally move one or more teeth.

### Embodiment O

The dental appliance as in one of embodiments A-N, wherein the shell comprises a polymer chosen from polyamide, polyethylene terephthalate, polybutylene terephthalate, polyester/polycarbonate copolymer, polyolefin, cyclic olefin polymer, styrenic copolymer, polyetherimide, polyetheretherketone, polyethersulfone, polytrimethylene terephthalate, parylene, and mixtures and combinations thereof.

### Embodiment P

The dental appliance as in one of embodiments A-O, wherein the coating has a thickness of about 1 μm to about 5 μm.

### Embodiment Q

The dental appliance as in one of embodiments A-P, wherein the coating has a thickness of about 3 μm.

### Embodiment R

A method of making a dental appliance, the method comprising:

forming a polymeric shell comprising a plurality of cavities in a first major surface thereof, wherein the cavities are configured to receive one or more teeth; and
applying a coating composition on the polymeric shell, wherein the coating composition comprises a polyester with an ion exchange functional group covalently bonded thereto, and wherein the ion exchange functional group is configurable to release in an oral environment at least one therapeutically beneficial ion at a surface of a tooth, and wherein the ion exchange functional group comprises a first metal ion.

### Embodiment S

The method of embodiment R, further comprising drying the coating composition to form a first coating on the polymeric shell.

### Embodiment T

The method of embodiment S, further comprising applying to the first coating an ionic solution with a second metal ion different from the first metal ion to replace the first metal ion on at least a portion of the ion exchange functional group with the second metal ion and form a second coating, wherein the second ion is a therapeutically beneficial ion chosen from calcium, fluoride, phosphate, and combinations thereof.

### Embodiment U

The method of embodiment T, further comprising drying the second coating to form a therapeutic coating on the polymeric shell and create the dental appliance, wherein the therapeutic coating is configured to supply in an oral environment a controlled release of at least one of the therapeutically beneficial ions at a surface of a tooth.

### Embodiment V

The method as in one of embodiments R-U, wherein the therapeutic coating is about 1 micron to about 5 microns thick.

### Embodiment W

The method as in one of embodiments T-V, wherein the therapeutically beneficial metal ion is calcium.

### Embodiment X

The method as in one of embodiments R-W, wherein the coating composition further comprises water and a surfactant.

### Embodiment Y

The method as in one of embodiments R-X, wherein the polyester comprises a sulfopolyester.

### Embodiment Z

The method as in one of embodiments R-X, wherein the polyester comprises a backbone having an aromatic nucleus with a metal sulfonate group RSO₃₋ attached thereto, and wherein R is a functional group chosen from hydroxy, carboxy, amino, and combinations thereof.

### Embodiment AA

The method of embodiment Z, wherein the first metal ion on the metal sulfonate group is a monovalent metal ion.

### Embodiment AB

The method of embodiment AA, wherein the monovalent metal ion is chosen from Na⁺, Li⁺, K⁺, Ag⁺ and mixtures and combinations thereof.

### Embodiment AC

The method of embodiment T, wherein the second metal ion is a divalent alkali metal ion.

### Embodiment AD

The method of embodiment AC, wherein the divalent alkali metal ion is chosen from Mg²⁺, Ca²⁺, Ni²⁺, Fe²⁺, Sr²⁺, Sn²⁺ and mixtures and combinations thereof.

### Embodiment AE

The method of embodiment AD, wherein the divalent alkali metal ion is Ca².

### Embodiment AF

The method as in one of embodiments R-AE, wherein the ion exchange compound comprises a quaternary ammonium compound chosen from tetraalkylammoniums, alkylated pyridines, alkylated immidazoles, and combinations thereof.

### Embodiment AG

The method of embodiment T, wherein the second metal ion is F.

### Embodiment AH

The method of embodiments R-AG, wherein the shell comprises a polymer chosen from polyamide, polyethylene terephthalate, polybutylene terephthalate, polyester/polycarbonate copolymer, polyolefin, cyclic olefin polymer, styrenic copolymer, polyetherimide, polyetheretherketone, polyethersulfone, polytrimethylene terephthalate, parylene, and mixtures and combinations thereof.

### Embodiment AI

The method as in one of embodiments R-AH, further comprising treating the surface of the polymeric shell prior to applying the coating composition.

### Embodiment AJ

The method as in one of embodiments R-AI, wherein the polymeric shell is formed by a three-dimensional printing process.

### Embodiment AK

The method as in one embodiments R-AJ, wherein the polymeric shell is formed by thermoforming a polymeric sheet to create the cavities.

### Embodiment AL

A method of making a dental appliance, the method comprising:

applying a coating composition on at least one major surface of a substantially flat sheet of a polymeric material, wherein the coating composition comprises a polyester with an ion exchange functional group covalently bonded thereto, wherein the ion exchange functional group is configurable to release in an oral environment at least one therapeutically beneficial ion at a surface of a tooth, and wherein the ion exchange functional group comprises a first metal ion; and
forming a plurality of cavities in the polymeric material to form a polymeric shell, wherein the cavities are configured to receive one or more teeth.

### Embodiment AM

The method of embodiment AL, further comprising treating the major surface prior to applying the coating composition.

### Embodiment AN

The method of embodiment AL or AM, further comprising drying the coating composition to form a first coating prior to forming the cavities.

### Embodiment AO

The method as in one of embodiments AL-AN, wherein the cavities in the polymeric material are thermally formed.

### Embodiment AP

The method as in one of embodiments AL-AO, further comprising:

applying to the first coating an ionic solution with a second metal ion, different from the first metal ion; and
replacing at least a portion of the first metal ions with a second metal ion to form a second coating, wherein the second metal ion is a therapeutically beneficial ion chosen from calcium, fluoride, phosphate, and combinations thereof.

### Embodiment AQ

The method as in one of embodiment AL-AP, further comprising drying the second coating to form a therapeutic coating on the polymeric shell and create the dental appliance, wherein the therapeutic coating supplies in an oral environment a controlled release of at least one of the therapeutically beneficial ions at a surface of a tooth.

### Embodiment AR

The method of embodiment AQ, wherein the therapeutic coating is about 1 micron to about 5 microns thick.

### Embodiment AS

The method as in one of embodiments AP-AR, wherein the therapeutically beneficial ion is calcium.

### Embodiment AT

The method as in one of embodiments AL-AS, wherein the coating composition further comprises water and a surfactant.

### Embodiment AU

The method as in one of embodiments AL-AT, wherein the polyester comprises a sulfopolyester.

### Embodiment AV

The method as in one of embodiments AL-AU, wherein the polyester comprises a backbone having an aromatic nucleus with a metal sulfonate group RSO₃₋ attached thereto, and wherein R is a functional group chosen from hydroxy, carboxy, amino, and combinations thereof.

### Embodiment AW

The method of embodiment AV, wherein the metal sulfonate group comprises a monovalent metal ion.

### Embodiment AX

The method of embodiment AW, wherein the monovalent metal ion is chosen from Na⁺, Li⁺, K⁺, NH₄⁺, Ag⁺, and mixtures and combinations thereof.

### Embodiment AY

The method as in one of embodiments AL-AX, wherein the ionic solution is chosen from CaCl₂, Ca(NO₃)₂, calcium gluconate, calcium gluconate lactate, and mixtures and combinations thereof.

### Embodiment AZ

The method as in one of embodiments AL-AY, wherein the second metal ion is a divalent alkali metal ion.

### Embodiment AAA

The method of embodiment AZ, wherein the divalent alkali metal ion is chosen from Mg²⁺, Ca²⁺, Ni²⁺, Fe²⁺, Sr²⁺, Sn²⁺, and mixtures and combinations thereof.

### Embodiment AAB

The method of embodiment AAA, wherein the divalent alkali metal ion is Ca²⁺.

### Embodiment AAC

The method as in one of embodiments AL-AAB, wherein the shell comprises a polymer chosen from polyamide, polyethylene terephthalate, polybutylene terephthalate, polyester/polycarbonate copolymer, polyolefin, cyclic olefin polymer, styrenic copolymer, polyetherimide, polyetheretherketone, polyethersulfone, polytrimethylene terephthalate, parylene, and mixtures and combinations thereof.

### Embodiment AAD

The method as in one of embodiments AM-AAC, wherein the polymeric material is chosen from polyethylene terephthalate, polyethylene terephthalate glycol, poly cyclohexylenedimethylene terephthalate glycol, and mixtures and combinations thereof.

### Embodiment AAE

A method of making a dental appliance, the method comprising:

applying a coating composition on at least one major surface of a substantially flat sheet of a polymeric material, wherein the coating composition comprises a polymer with an ion exchange functional group covalently bonded thereto, wherein the polymer comprises a polyester, wherein the polymer comprises a quaternary ammonium compound chosen from tetraalkylammoniums, alkylated pyridines, alkylated immidazoles, and combinations thereof, and wherein the ion exchange functional group is configurable to release in an oral environment at least one therapeutically beneficial ion at a surface of a tooth, and wherein the ion exchange functional group comprises a first metal ion; and
forming a plurality of cavities in the polymeric material to form a polymeric shell, wherein the cavities are configured to receive one or more teeth.

### Embodiment AAF

The method of embodiment AAD, wherein the second metal ion is F−.

### Embodiment AAG

A method of treating demineralization of a surface of a tooth, the method comprising positioning a dental appliance adjacent to the surface of the tooth, wherein the dental appliance comprises a polymeric shell with a plurality of cavities configured to incrementally move one or more teeth, and wherein the polymeric shell comprises a coating thereon, the coating comprising a sulfopolyester with an ion exchange functional group covalently bonded thereto, and wherein the ion exchange functional group supplies in an oral environment at least one therapeutically beneficial ion at a surface of a tooth.

### Embodiment AAH

The method of embodiment AAF, wherein the polymer supplies cations at the surface of the tooth.

### Embodiment AAI

The method of embodiment AAF or AAG, wherein the polymer supplies anions at the surface of the tooth.

### Embodiment AAJ

The method as in one of embodiments AAF-AAH, wherein the therapeutically beneficial ion supplied at the surface of the tooth is chosen from calcium, fluoride, phosphate and combinations thereof.

### Embodiment AAK

The method as in one of embodiments AAF-AAI, wherein the therapeutically beneficial ion supplied at the surface of the tooth is calcium.

### Embodiment AAL

The method as in one of embodiments AAF-AAJ, wherein the polymer comprises at least one of a metal sulfonic group and a quaternary ammonium group.

### Embodiment AAM

A method of treating demineralization of a tooth, the method comprising: providing a polymeric shell with a plurality of cavities configured to incrementally move one or more teeth, wherein the polymeric shell comprises a coating thereon, the coating comprising a sulfopolyester with an ion exchange functional group, wherein the ion exchange functional group comprises a first metal ion;

applying to the first coating an ionic solution with a second metal ion, different from the first metal ion; replacing the first metal ion on at least a portion of the ion exchange functional group with the second metal ion to form a second coating, wherein the second ion is a therapeutically beneficial ion chosen from calcium, fluoride, phosphate, and combinations thereof; and
positioning the second coating such that the second coating is adjacent to a surface of at least one tooth, the second coating releasing in an oral environment at least one therapeutically beneficial ion at the surface of the tooth.

### Embodiment AAN

The method of embodiment AAL, wherein the ionic solution comprises calcium ions.

### Embodiment AAO

The method of embodiment AAM, wherein the ionic solution is CaBr₂.

### Embodiment AAP

A kit comprising:

a dental appliance comprising a polymeric shell with a plurality of cavities configured to incrementally move one or more teeth, wherein the polymeric shell comprises a coating thereon, the coating comprising a sulfopolyester with an ion exchange functional group covalently bonded thereto, and wherein the ion exchange functional group releases in an oral environment at least one therapeutically beneficial ion at a surface of a tooth; and
a solution comprising ions to periodically replenish the therapeutically beneficial ions of the coating.

### Embodiment AAQ

The kit of embodiment AAO, further comprising a storage tray for the dental appliance, wherein the storage tray comprises a well to store the dental appliance and an amount of the solution sufficient to replenish the therapeutically beneficial ions.

### Embodiment AAR

The kit of embodiment AAO or AAP, further comprising instructions for use of the dental appliance and the solution.

### Embodiment AAS

The kit as in one of embodiments AAO-AAQ, wherein the ion supplied at the surface of the tooth is chosen from calcium, fluoride, phosphate and combinations thereof.

### Embodiment AAT

The kit as in one embodiments AAO-AAR, wherein the ion supplied at the surface of the tooth is calcium.

### Embodiment AAU

The kit as in one embodiments AAO-AAS, wherein the solution is an aqueous solution comprising calcium ions.

### Embodiment AAV

The kit of embodiment AAT, wherein the solution is a 1% aqueous CaCl₂ solution.

### Embodiment AAW

The kit of embodiment AAT, wherein the solution is a 10% aqueous CaCl₂ solution.

### Embodiment AAX

A dental appliance comprising:

a polymeric shell with an arrangement of cavities configured to receive one or more teeth; and a coating on at least portion of the polymeric shell, wherein the coating comprises a polymer with an ion exchange functional group covalently bonded thereto, wherein the polymer comprises a quaternary ammonium compound chosen from tetraalkylammoniums, alkylated pyridines, alkylated immidazoles, and combinations thereof, and wherein the ion exchange functional group releases in an oral environment at least one therapeutically beneficial ion at a surface of a tooth.

1. A dental appliance comprising:
a polymeric shell with an arrangement of cavities configured to receive one or more teeth; and
a coating on at least portion of the polymeric shell, wherein the coating comprises a polyester with an ion exchange functional group covalently bonded thereto, and wherein the ion exchange functional group releases in an oral environment at least one therapeutically beneficial ion at a surface of a tooth.

2. The dental appliance of claim 1, wherein the ion exchange functional group supplies anions at the surface of the tooth.

3. The dental appliance of claim 1, wherein the ion supplied at the surface of the tooth is chosen from calcium, fluoride, phosphate and combinations thereof.

4. The dental appliance of claim 1, wherein the polyester comprises a sulfopolyester.

5. The dental appliance of claim 1, wherein the polyester comprises a backbone having an aromatic nucleus with a metal sulfonate group RSO₃₋ attached thereto, wherein R is a functional group chosen from hydroxy, carboxy, amino, and combinations thereof.

6. The dental appliance of claim 5, wherein the metal in the metal sulfonate group is chosen from Na⁺, Li⁺, K⁺, Mg⁺, Ca²⁺, Ni²⁺, Fe²⁺, Fe³⁺, Zn²⁺, Sr²⁺, Ag⁺, Sn²⁺, an ammonium substituted with an alkyl or hydroxy alkyl radical having 1 to 4 carbon atoms, and combinations thereof.

7. A method of making a dental appliance, the method comprising:
forming a polymeric shell comprising a plurality of cavities in a first major surface thereof, wherein the cavities are configured to receive one or more teeth; and
applying a coating composition on the polymeric shell, wherein the coating composition comprises a polyester with an ion exchange functional group covalently bonded thereto, and wherein the ion exchange functional group is configurable to release in an oral environment at least one therapeutically beneficial ion at a surface of a tooth, and wherein the ion exchange functional group comprises a first metal ion.

8. The method of claim 7, further comprising drying the coating composition to form a first coating on the polymeric shell.

9. The method of claim 8, further comprising applying to the first coating an ionic solution with a second metal ion different from the first metal ion to replace the first metal ion on at least a portion of the ion exchange functional group with the second metal ion and form a second coating, wherein the second ion is a therapeutically beneficial ion chosen from calcium, fluoride, phosphate, and combinations thereof.

10. The method of claim 9, further comprising drying the second coating to form a therapeutic coating on the polymeric shell and create the dental appliance, wherein the therapeutic coating is configured to supply in an oral environment a controlled release of at least one of the therapeutically beneficial ions at a surface of a tooth.

11. The method of claim 6, wherein the polyester comprises a sulfopolyester.

12. A kit comprising:
a dental appliance comprising a polymeric shell with a plurality of cavities configured to incrementally move one or more teeth, wherein the polymeric shell comprises a coating thereon, the coating comprising a sulfopolyester with an ion exchange functional group covalently bonded thereto, and wherein the ion exchange functional group releases in an oral environment at least one therapeutically beneficial ion at a surface of a tooth; and
a solution comprising ions to periodically replenish the therapeutically beneficial ions of the coating.

13. The kit of claim 12, further comprising a storage tray for the dental appliance, wherein the storage tray comprises a well to store the dental appliance and an amount of the solution sufficient to replenish the therapeutically beneficial ions.

14. The kit of claim 12, wherein the ion supplied at the surface of the tooth is chosen from calcium, fluoride, phosphate and combinations thereof.

15. The kit of claim 12, wherein the solution is an aqueous solution comprising calcium ions.
